(19) United States
(12) Patent Application Publication
(10) Pub. No.: US 2015/0313462 A1
(43) Pub. Date: Nov. 5, 2015

(54) Method and System of using Photorefractive effects to examine eyes using a portable device

(71) Applicant: Alexander Reis, Eschen (LI)

(72) Inventor: Alexander Reis, Eschen (LI)

(21) Appl. No.: 14/269,186

(22) Filed: May 4, 2014

(51) Int. Cl. A61B 3/103; A61B 3/14

(52) U.S. Cl. CPC A61B 3/103; A61B 3/14

(57) ABSTRACT

The current invention involves the use of portable devices such as a smart phone or tablet to perform an examination and diagnostic of an eye or pair of eyes using a photorefraction system and technique. The portable device would send a ray of light through the cornea and take measurements of the refraction. It would then use this information to diagnose any defects of the eye using a software application (app) or program or it can transmit the information to a server on which the diagnostic software is located where the server will run the diagnostic analysis.

### CROSS-REFERENCES TO RELATED APPLICATIONS

None

### FIELD OF THE INVENTION

This device refers to the field of a Method and System to measure the refraction of an eye in more particular using a hand held device.

### BACKGROUND OF THE INVENTION

Optometry is a health care profession concerned with the health of the eyes. Optometrists are trained to prescribe and fit lenses to improve vision, and to diagnose and treat various eye diseases.

Normally, corrective lenses are used to correct refractive errors by bending the light entering the eye in order to alleviate the effects of conditions such as myopia and hyperopia.

Myopia, which is also known as nearsightedness, is where the light that comes into the eye does not directly focus on the retina but in front of it, causing the image that one sees when looking at a distant object to be out of focus, but in focus when looking at a close object.

Hyperopia, also known as farsightedness or hyperopia, is a defect of vision caused by an optical imperfection in the eye such as where the eyeball is too short or the lens cannot become round enough, causing difficulty focusing on near objects, and in extreme cases causing a sufferer to be unable to focus on objects at any distance. When an object moves toward the eye, the eye must increase its optical power to keep the image in focus on the retina. In hyperopia, the power of the cornea and lens is insufficient so the image will appear blurred.

These conditions are detected through examination and diagnosis of an eye specialist such as an Optician, Optometrist or Ophthalmologist.

Photorefraction, invented in the 1970;s, is a method to measure the refraction of an eye by using a photograph of the red reflex of an eye. Photorefraction devices use this method to measure the refraction of an eye by analyzing the pupils red reflex using a photograph of the pupil. The core of the principle is analyzing the red reflex of an eye. Up to now, there are only two devices using this principle, both are handheld and expensive. They can cost up to $10,000 dollars or more making it cost prohibitive to treat patients. The cost of these devices make them less accessible to those in rural areas and in developing nations.

More on photorefraction is explained in an article, Simulation of eccentric photorefraction images, by Ying-Ling Chen, Bo Tan, and J. W. L. Lewis, Center for Laser Applications, The University of Tennessee Space Institute, 411 B. H. Goethert Parkway, Tullahoma, Tenn. 37388-8897, 14 Jul. 2003/Vol. 11, No. 14/OPTICS EXPRESS 1628 which is incorporated by reference.

There is a need to make the examination and diagnostics for eye defects simpler, cheaper and easier to use as well as being portable.

There remains room for improvement in the art.

### SUMMARY OF THE INVENTION

The current invention is a Method and System of using Photorefractive effects to examine eyes using a portable device.

In operation the user would use a portable device such as a smart phone, tablet or similar device to perform an examination and diagnostic of an eye or pair of eyes using a photorefraction system and technique. The portable device would send a ray of light through the cornea and take measurements of the refraction. It would then use this information to diagnose any refractive error of the eye using a software application (app) or program or it could transmit the information to a server on which the diagnostic software is located wherein the server would run the diagnostic analysis.

The current invention can also use a photograph of an eye with the red reflex, where the picture is scanned and run through the analyzing software. With the current invention there is no need to build a specially designed device to use photorefraction.

### BRIEF DESCRIPTION OF DRAWINGS

Without restricting the full scope of this invention, the preferred form of this invention is illustrated in the following drawings in which:

FIG. 1 shows the conditions such as myopia and hypermetropia;

FIG. 2 shows a portable device being used; and

FIG. 3 shows multiple devices being connected to a server; and

FIG. 4 shows the system using a scanned picture.

### DETAILED DESCRIPTION OF THE PREFERRED EMBODIMENT

There are a number of significant design features and improvements incorporated within the invention.

While Photorefraction was invented in the 1970s, and is a method to measure the refraction of an eye by analysing the pupils red reflex using a photograph of the pupil, prior to the current invention there were only two devices using this method. Both are handheld and expensive and are infrared base. The current invention is a method to transfer this principle to any handheld-device, such as a smartphone, tablet or laptop computer. This method can be used with any device with a camera and a microprocessor or web connection.

In other words, with the current invention there is not a need to use and build a specially designed device to use photorefraction making it much easier to analyze the reflex from the pupil. This is a novel concept that is a vast improvement over prior art.

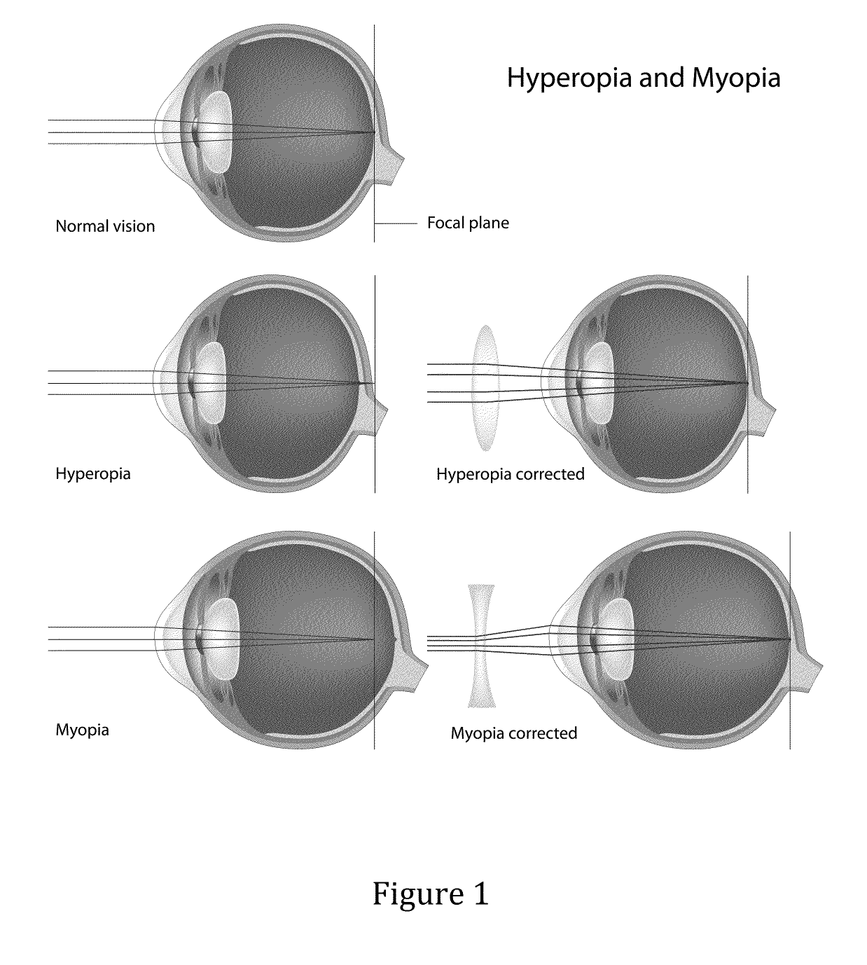

The current invention will use photorefractive effects to measure and diagnose whether or not the eyes are mopic, hyperopic or astigmatic, as well as other issues with the eyes that may require corrective measures using a hand held or mobile device, such as a cell phone, tablet, camera or laptop. Some of the main refractive errors being examined for are conditions such as myopia, hypermetropia and astigmatism. These conditions are shown in FIG. 1.

Myopia is where the light that comes in does not directly focus on the retina but in front of it, causing the image that one sees when looking at a distant object to be out of focus, but in focus when looking at a close object.

Hyperopia is a defect of vision caused by an imperfection in the eye such as where the eyeball is too short or the lens cannot become round enough, causing difficulty focusing on near objects, and in extreme cases causing a sufferer to be unable to focus on objects at any distance.

Photorefraction can be used for the detection of specific vision problems. The preferred embodiment is off-axis, or eccentric, photorefraction for which an image of the subject's pupil is obtained using a device, such as a camera, smart phone, tablet or similar device (such as a laptop), that is aligned eccentric to a flash-lamp illumination source. The geometric form and irradiance of pupil image will be dependent upon the subject's pupil size, refractive errors, staring angle, other properties of the eye, and the design parameters of the optical measurement system. The device can perform binocular measurements. This, of course, enables the testing of both eyes at the same time and measurably improves the ability to detect strabismus.

In the preferred embodiment the process is performed in a dimmed room to ensure the pupils are naturally dilated.

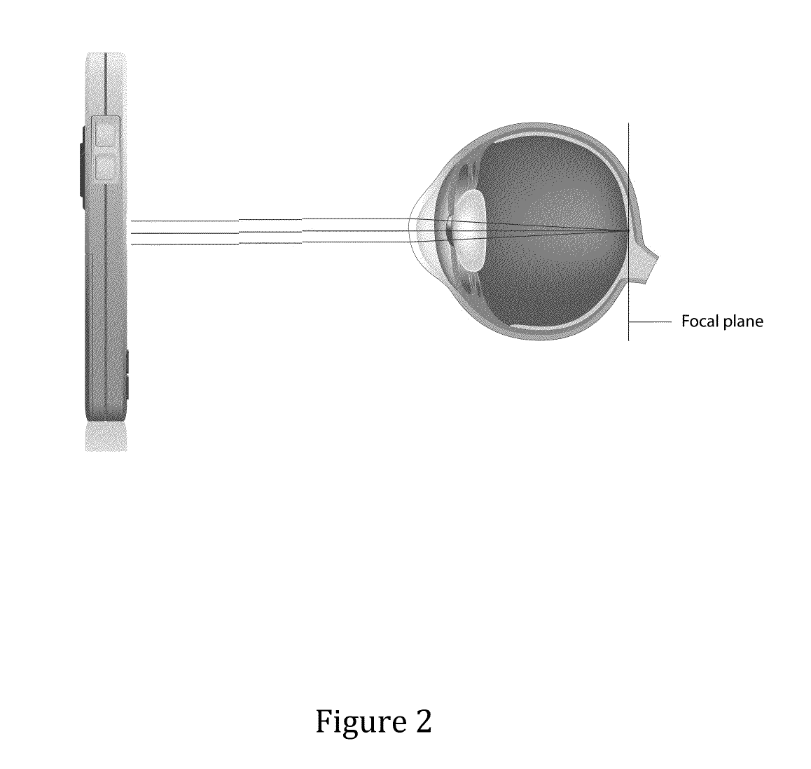

As shown in FIG. 2, the basic optical concept of photorefraction is that the light rays from the device enter the eye, are reflected from the retina, and are then collected by the device. With a properly functioning eye, the light rays from the far zone are imaged onto the retina into the smallest size. The reflected (or scattered) light rays from the retina are then focused by the eye in the second pass and form a converging cone for myopic or diverging cone if hyperopic, symmetric to the axis that connects the eye and light source. Normally, the signal light cone from an emmetropic eye has the smallest solid angle. The solid angle of the reflection light cone increases with the degree of refractive error.

Operations

The current invention can be used for measurement of refraction from a distance (80 cm-5 m). The setup is simple: it consists of a device (smart phone, camera, laptop, tablet or similar device) focused to the subject's pupils and a light source from the device or mounted close to the device aperture.

The limited size of the device aperture generates a (more or less well defined) papillary brightness distribution (crescent) that is characteristic for the eye's refraction. In the preferred embodiment, both eyes are refracted simultaneously.

For a measurement of astigmatic refractions (sphere, cylinder, angle of cylinder axis) at least 2 or 3 measurements with different orientations of the light source are necessary to be performed.

The device 10 using a program or an application (app) will measure the spherical aberration, the anisotropic scattering function of the retina, multiple scattering in the retinal tissue and the non-circular shape of the pupil.

The device will be easy to position properly given its small size and portable nature. The device's device 10 will record the photorefractive effects on the eye, or eyes if both are being measured. It will record this information as data.

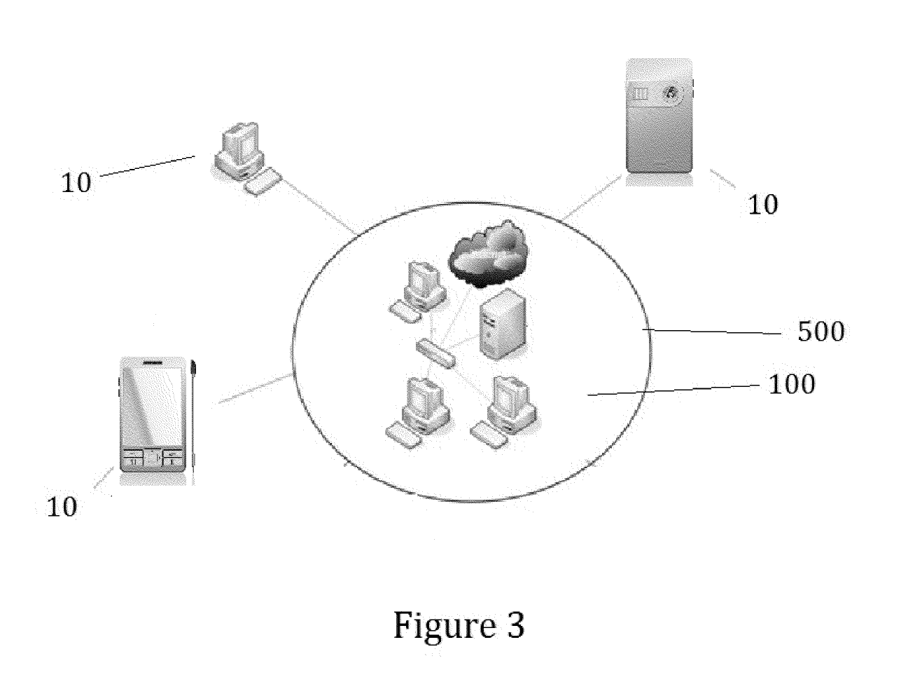

The device can use the measurement to calculate the findings itself using diagnostic software or application (app) if it has computing means such as a microprocessor with memory or it could be connected and upload the data to a server or servers 500 that have software 100 stored and run on electronic memory to analyze the results of the eye measurement to determine the findings. As shown in FIG. 3 multiple devices can be connected and transmit and receive data from the server 500. The server 500 can even be a world away in cases where the diagnostics are being done in 3^{rd} world countries.

The current invention uses smart algorithms to analyze the pictures taken by any portable device 10 with a camera and either an internet-connection or an onboard computer. This is novel and new to the art.

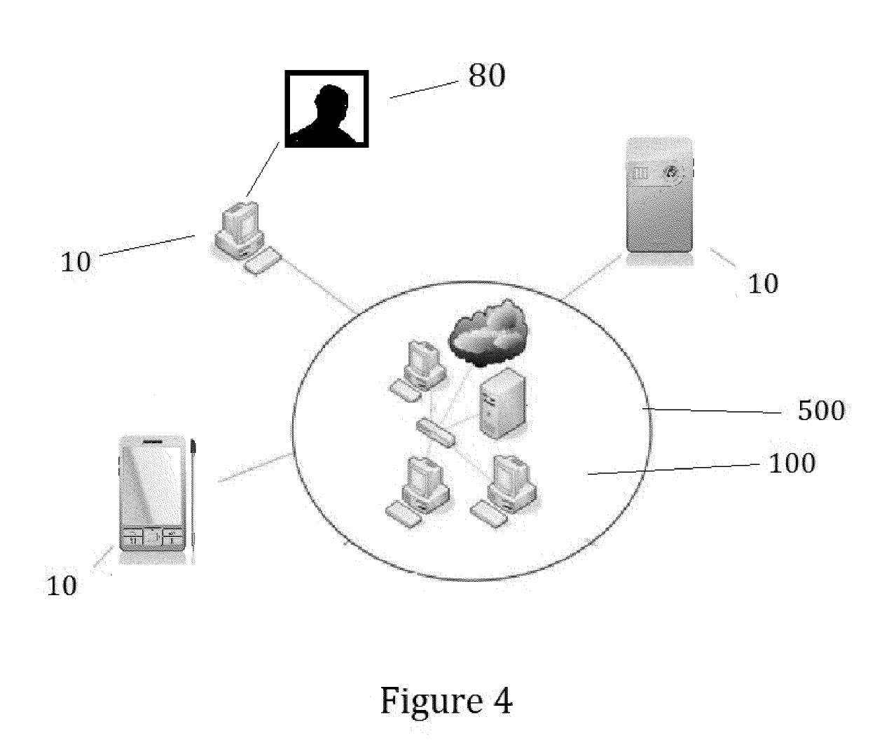

A user can also make a photographs 80 of an eye with the red reflex, scan the photographs 80, transmit the electronic image and run it through the analyzing software used above as shown in FIG. 4.

Advantages

The Inventor has created a system that can be used anywhere to efficiently and economically measure for and detect eye defects. This system will vastly increase the ability to perform and diagnose eye defects, especially in poorer regions of the world.

All description giving is for clarification purposes only, and not intended to limit the invention features and embodiment measurements. Further aspects of the invention will become clear from consideration of the drawings and the ensuing description of the preferred embodiments. A person skilled in the art will realize that other embodiments of the invention can vary and the details of the invention can be modified in a number of respects, all without departing from the inventive concept. Thus, the following drawings and descriptions are to be regarded as illustrative in nature and not restrictive.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur by those skilled in the art, it is not desired to limit the invention to the exact construction and operation shown and described, and accordingly, all suitable modifications and equivalents may be resorted to, falling within the scope of the invention.

1. A process to examine eyes comprising:
Using a portable device to generate readings to diagnose eyes using photorefraction by having light rays in the visible light spectrum from a light source enter the eye, reflected from the retina, and then collected by the device which will record the photorefractive effects on the eye and record this information as data which is used by a program to measure the spherical aberration, the anisometropic scattering function of the retina, multiple scattering in the retinal tissue and the non-circular shape of the pupil.

2. A process according to claim 1 further comprising having said device having a camera.

3. A process according to claim 1 further comprising having said device having an internet connection.

4. A process according to claim 1 further comprising having said device having a computing means.

5. A process according to claim 1 further comprising having said device taking pictures of the light refraction from the eye.

6. A process according to claim 1 further comprising having said device taking multiple pictures of the light refraction from the at different angles.

7. (canceled)

8. (canceled)

9. A process according to claim 1 further comprising where the readings are transmitted to a server.

10. A process according to claim 9 further comprising having a program on said server to analyze the readings.

11. A process according to claim 1 where readings from both eyes are taken at the same time.

12. A process according to claim 1 where said device produces a photograph which is then scanned and transmitted to a computing device where it is analyzed.

13. A process according to claim 1 where said device is a Smartphone.

14. A process according to claim 1 where said device is a camera.

15. A process according to claim 1 where said device is a tablet.

16. A process according to claim 1 where said device is a laptop.

17. A process according to claim 1 where both eyes are refracted simultaneously.

18. A process according to claim 1 further comprising having the program run on the device.
